(19) United States
(12) Patent Application Publication
(10) Pub. No.: US 2020/0246045 A1
(43) Pub. Date: Aug. 6, 2020

(54) TRIPARTITE MEDICAL INSTRUMENT OR PART

(71) Applicant: GRANIT MEDICAL INNOVATION, LLC, NEW YORK, NY (US)

(72) Inventor: Naomi L. Nakao, NEW YORK, NY (US)

(21) Appl. No.: 16/269,364

(22) Filed: Feb. 6, 2019

(51) Int. Cl. A61B 17/34; A61B 1/018; A61B 1/00; A61L 29/02

(52) U.S. Cl. CPC A61B 17/3478; A61B 1/018; A61B 2017/0034; A61B 17/3417; A61L 29/02; A61B 1/00133

(57) ABSTRACT

A medical instrument insertable through a catheter or a working channel of an endoscope exemplarily for performing a needle biopsy on a patient's internal body tissues comprises an elongate member having a proximal end portion, a distal end portion and a connector between the proximal end portion and the distal end portion and bonded thereto. A hollow needle tip may be provided at the distal end, the instrument may be tubular, at least a distal end, and the instrument may be inserted in a sheath member.

### FIELD OF THE INVENTION

This invention relates to a medical instrument that may be used in minimally invasive procedures. The instrument or device is exemplarily configured for insertion through or deployment via a working channel of a flexible endoscope.

An instrument in accordance with this invention is utilizable with ultrasound, and in some cases an ultrasound-endoscope, for performing a trans-luminal needle biopsy through an endoscope, on a patient's internal body tissues at a surgical site not visible to the unaided eye.

The instrument of this invention facilitates the performance of a fine needle biopsy (FNB)—obtaining a core of tissue, instead of a fine needle aspiration (FNA)—obtaining a number of singular cells aspirated from morselized tissue. Obtaining an entire core biopsy of tissue is made possible due to the rotating handle design of the invention in combination with the special configuration of the elongate flexible needle assembly, such that when deployed, the needle is plunged into the lesion to be sampled, advancing both in an axial and a rotary fashion.

However, a flexible instrument pursuant to the invention has other applications where a distal end portion of the instrument is flexible but sustains its shape despite repeated deforming strains.

### BACKGROUND OF THE INVENTION

FNA has been a well-accepted method for obtaining tissue samples for histologic analysis in diagnosing tumors of the pancreas and other extra-luminal soft tissue organs through endoscopic ultrasound (EUS) and EUS-guided fine needle aspiration (EUS-FNA).

Conventional surgical techniques for obtaining tissue samples accessible only through a flexible ultrasound-endoscope using a fine needle generally require numerous (15-30) needle plunges. Collectively called “the woodpecker technique”, these numerous needle plunges cause morselization, or liquefaction of a tumor lesion enabling subsequent aspiration of cells through the needle of an FNA instrument. These procedures often result in obtaining a small number of cells, which may or may not be diagnostic. Such procedures are often traumatic due to the multiple needle plunges necessary to liquefy and aspirate cells. This is especially true in the case of pancreatic biopsies, the pancreas being a vascular organ. Liquification of a pancreatic tumor by means of multiple needle sticks may cause a pancreatic cancer to disseminate. In addition, trauma to the pancreas, it being an organ that secretes digestive enzymes, may cause acute pancreatitis.

The current technique used during EUS-FNA of a pancreatic tumor entails passing an 18-22 gauge stainless steel needle into a suspected tumor. This needle is passed through the working channel of a linear echo endoscope under real-time guidance into the endo-sonographically visualized pancreatic mass. The needle is moved back and forth into the lesion to be sampled, while suction is being applied. The specimens obtained are subsequently placed onto a slide, and immediately examined by a pathologist, who must be present during every such procedure.

Aspirating a sample through a needle from a fluid medium a simple procedure. Aspirating a sample from a solid mass is difficult. It is for this reason that multiple needle sticks are necessary to morcelize the tumor before aspirating. Most pancreatic EUS-FNA procedures take up to 30 needle passes to make a definitive cytologic diagnosis of pancreatic carcinoma. Oftentimes, the only cells that are obtained are blood cells or normal pancreatic tissue cells. In such a case, the procedure is repeated until one or more cancer cells are detected by the pathologist.

U.S. Pat. No. 7,722,549 to Naomi Nakao discloses an instrument that includes a rotating needle for taking core biopsies of extra-luminal internal organs. Even when the needle of this invention is of a fine gauge, a single, gentle needle pass yields an excellent core biopsy specimen. Pursuant to the invention of U.S. Pat. No. 7,722,549, the needle automatically rotates while being advanced into a lesion, thus garnishing a solid core biopsy at every needle pass.

One other problem with conventional EUS-FNA instruments as used during endoscopic tissue aspiration procedures is the unavoidable bending and subsequent deformation of the stainless steel needle. The distal end of the needle assumes the curvilinear shape of the endoscope while it traverses the tortuous duodenal sweep. This needle deformation hinders the operator's aim at the tumor, causing poor tissue sampling. In order to address this problem, the makers of one EUS-FNA instrument use an elongate tubular needle shaft made entirely of nitinol, a metal alloy possessing shape memory. Unfortunately, using such a long nitinol tube substantially increases the cost, more than doubling the price of the instrument.

### OBJECTS OF THE INVENTION

An object of the present invention is to provide an improved medical instrument of the above-described type.

It is another object of the present invention to provide such a medical instrument that is substantially reduced in cost.

A more particular object of the present invention is to provide a fine-diameter elongate member medical device, exemplarily utilizable as a fine needle in endoscopic procedures.

Another object of the invention is to facilitate the manufacture of a flexible fine diameter, tubular medical device consisting of an elongate proximal portion made of stainless steel, coupled at a distal end with a relatively shorter and therefore less costly flexible tubular nitinol needle, rendering the entire device considerably less costly than an all nitinol device.

These and other objects of the invention will be apparent from the drawings and descriptions herein. Although every object of the invention is deemed to have been met by at least one embodiment of the invention, there is not necessarily any one embodiment that achieves all of the objects of the invention.

### SUMMARY OF THE INVENTION

This invention facilitates fine needle biopsy (FNB), such as that disclosed in U.S. Pat. No. 7,722,549, by providing a needle member that is rotatable as it is being advanced, so that when plunged into a target tissue it moves forward axially and rotatably, that does not assume an arcuate deformation upon being passed along the tortuously curved path of an endoscope's working channel.

A medical instrument in accordance with the present invention comprises an elongate shaft member having a diameter sufficiently small so that the shaft member is disposable in a channel of a flexible endoscope, the elongate shaft member including a first elongate segment made of stainless steel, a second elongate segment made of nitinol, and a connector element made of nickel. The connector element is bonded on one side to an end of the first elongate segment and on an opposite side to an end of the second elongate segment.

Pursuant to another feature of the present invention, the medical instrument further comprises an elongate tubular sheath member, the elongate shaft member being disposable within the elongate tubular sheath member, the elongate tubular sheath member having a diameter sufficiently small so that the elongate tubular sheath member is disposable in a channel of a flexible endoscope.

In accordance with a further feature of the present invention, the first elongate segment, the second elongate segment and the connector are all tubular elements. In addition, the first elongate segment and the second elongate segment are flexible elements. In a preferred embodiment, the two elongate segments and the connector together exhibit a smoothly continuous internal lumen of uniform diameter. Moreover, the external diameter of the entire elongate shaft member has a uniform or constant size.

According to another feature of the present invention, the elongate shaft member is coupled with a handle assembly as disclosed in U.S. Pat. No. 7,722,549. In a preferred application of the present invention, the elongate shaft member, when deployed, performs such that the distal needle end rotates and axially advances in a 1:1 fashion with rotation of a handle actuator, yet the entire shaft member is made of materials much less costly than if it were wholly made of the shape memory metal, nitinol.

Pursuant to yet another feature of the present invention, the connector element is heat bonded or welded on the one side to the first elongate segment and on the opposite side to the second elongate segment.

The second elongate segment may be provided at a free end, opposite the connecting member, with a needle configuration. In this case the instrument may be used in an endoscope assembly disclosed in U.S. Pat. No. 7,722,549 for implementing the procedures detailed in that document.

The present invention may be used in other minimally invasive medical procedures, for instance, in transcutaneous intravascular procedures with the tri-metallic instrument inserted into a patient via a catheter. Guide wires manipulated to direct catheters through meandering internal body spaces may also benefit from the present invention.

A fine-diameter medical device in accordance with the present invention facilitates obtaining core tissue biopsies, because the device is not subject to warping or deformation owing to its being passed through and along an arcuate path, and because the fine-diameter elongate member is rotatable, concurrent 1:1 with the rotation effected by the handle, a feat heretofore only accomplished by an all nitinol needle shaft, and yet, in accordance with this invention, the fine-diameter medical device is substantially less costly than endoscopic needles made entirely of nitinol.

A fine-diameter medical needle in accordance with the present invention is useful for collecting a core tissue biopsy from internal tissues situated extra-lumenally to the gastrointestinal track. The device of this invention may also be used for lung biopsies in order to sample lesions that are located extra-lumenally to the trachea (The EBUS Procedure). The needle assembly requires only a minimal number of gentle needle passes into the organ tissue, thereby reducing undue trauma to the pancreas or other internal organs that are sampled, avoiding possible tumor dissemination, and enabling a manufacturing method whereby the entire instrument may be purchased at approximately half the price of competitive devices.

### BRIEF DESCRIPTION OF THE DRAWINGS

FIG. 1 is a schematic perspective view of an endoscopic instrument assembly for use in obtaining a tissue sample from an internal organ of a patient via a trans-luminal procedure.

FIG. 2A is a side elevational view, on an enlarged scale, of a camming collar or tube provided at the distal end of the endoscopic instrument assembly of FIG. 1.

FIG. 2B is a side elevational view, on an enlarged scale, of a distal tip of a needle element of the endoscopic instrument assembly of FIG. 1.

FIG. 2C is a side elevational view, on a smaller scale, of a distal end portion of the needle element of FIG. 2B, showing the needle element provided with a laterally projecting guide stub.

FIG. 2D is a partial longitudinal cross-sectional view of the distal end portion of the endoscopic instrument assembly of FIG. 1, showing the components of FIGS. 2A-2C.

FIG. 3 is a schematic perspective view of the distal portion of the assembly of FIG. 1 in a pre-deployment position, as introduced through a flexible endoscope.

FIG. 4 is a schematic perspective of the distal portion of the assembly of FIG. 1 with the needle element of FIGS. 2B-2D starting to be deployed from its housing.

FIG. 5 is a schematic perspective view of the needle of FIG. 4 being deployed from its housing and introduced into a patient's body tissue.

FIG. 6 is a schematic view of the needle of FIGS. 4 and 5 interacting with a threaded collar so as to be turned inside the patient's tissue to obtain a core biopsy.

FIG. 7 is a schematic view of the needle being withdrawn into its housing with the specimen inside.

FIG. 8 is a schematic perspective view of a handle or actuator assembly of an endoscopic instrument assembly for core tissue sampling, with which a tripartite tubular needle device (see FIG. 16) in accordance with the present invention may be used.

FIG. 9 is a longitudinal cross-sectional, on an enlarged scale, of the handle or actuator assembly of FIG. 8.

FIG. 10 is a longitudinal cross-sectional view, on a scale smaller than that of FIG. 9, of a slotted or grooved metal plunger tube included in the handle or actuator assembly of FIGS. 8 and 9.

FIG. 11 is perspective view, on a greatly enlarged scale, of a key member or cam follower included in the handle or actuator assembly of FIGS. 8-10.

FIG. 12 is a side elevational view, on a reduced scale, of an endoscopic instrument assembly in which a tubular needle device in accordance with the present invention may be used.

FIG. 13 is a perspective view of a modified handle or actuator assembly of a core tissue sampling instrument.

FIG. 14 is a side elevational view of another endoscopic instrument assembly for core tissue sampling.

FIG. 15 is a schematic view of an upper portion of a person's digestive tract, showing a step in an endoscopic procedure.

FIG. 16 is partially a longitudinal cross-sectional view and partially a side elevational view of a medical instrument in accordance with the present invention.

### DETAILED DESCRIPTION

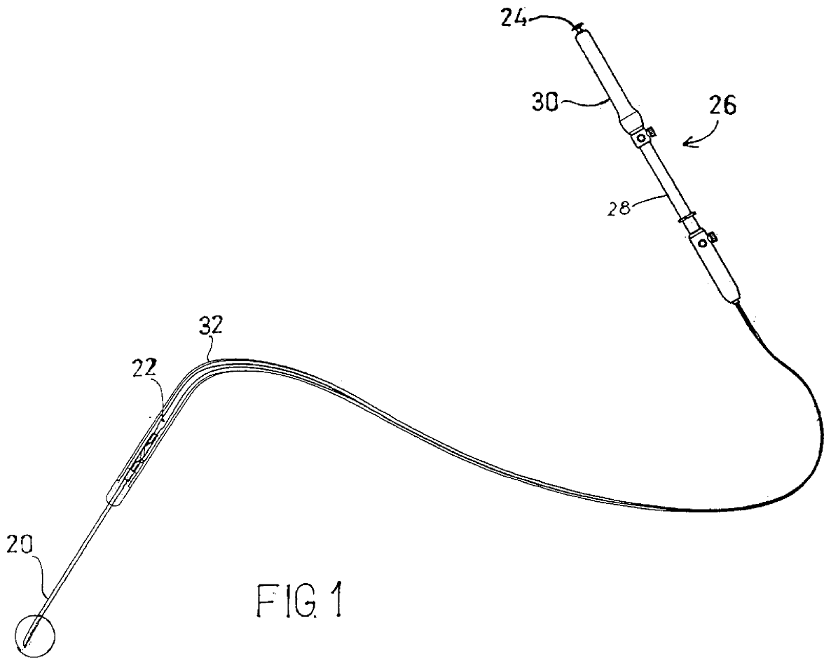

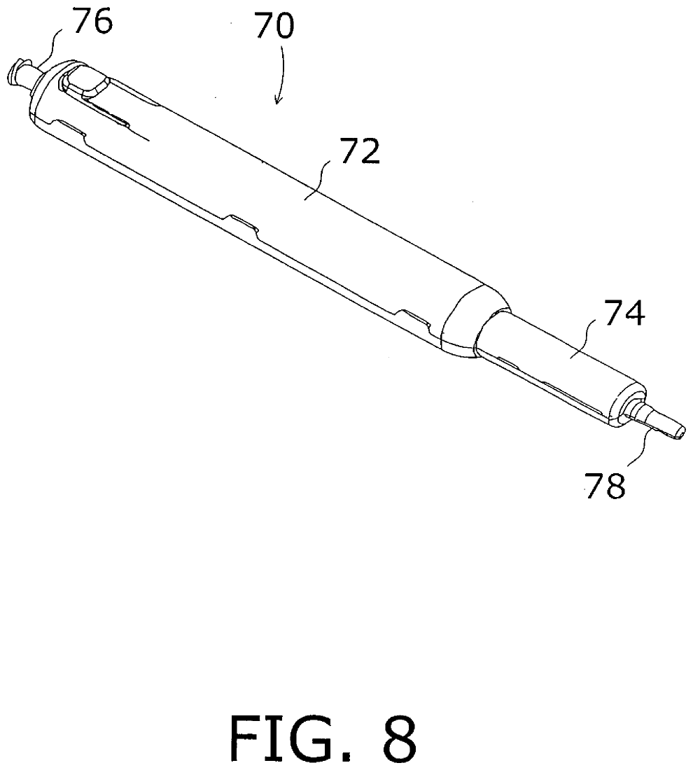

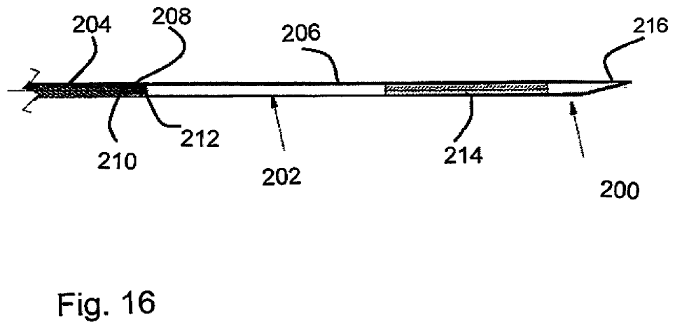

The present invention pertains to an improvement discussed hereinafter with reference to FIG. 16. The tubular needle device of that drawing figure may be incorporated for use with one or more of the assemblies disclosed in FIGS. 1-15, particularly FIGS. 8-12, in substitution for a shaft and needle elements thereof.

As illustrated in FIG. 1, a medical instrument assembly for use in procedures for obtaining tissue samples comprises a hollow needle element 20 communicating with a lumen (not shown) of a tubular shaft member 22 to enable suction of a patient's tissue to be obtained by needle 20. Tubular shaft member 22 is provided at a proximal end with a port 24 that communicates with the lumen of the tubular shaft member. As disclosed below with reference to other embodiments, needle element 20 may be an integral distal tip of shaft member 22.

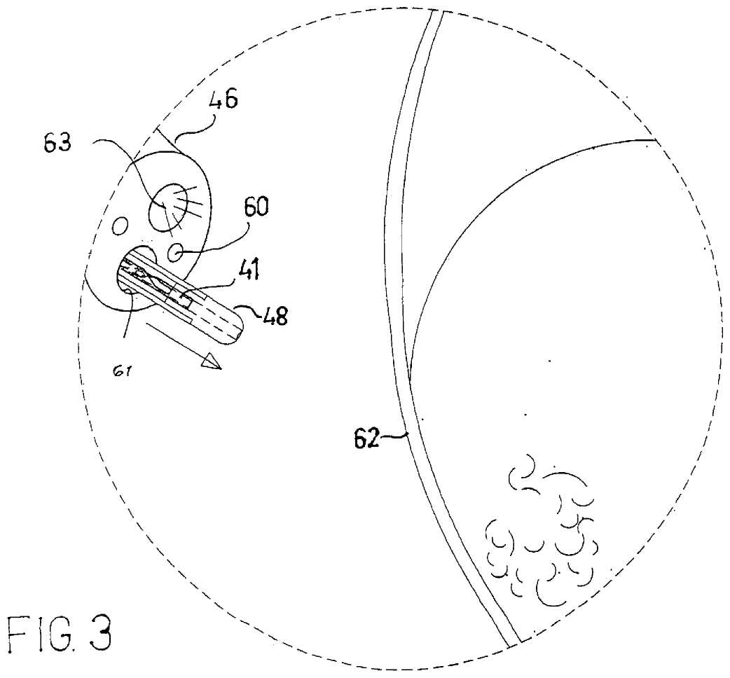

The instrument assembly of FIG. 1 includes an actuator subassembly 26 including a cylindrical body portion 28 carrying a slidably mounted shifter 30. Shifter is connected to tubular shaft member 22 for moving the tubular shaft member alternatively in the distal direction and the proximal direction through a flexible tubular sheath 32 that is fixed at a proximal end to cylindrical body portion 28. Sheath 32 has a sufficiently small diameter to enable insertion of the sheath, together with tubular shaft member 22 into a biopsy channel 61 of an endoscope insertion member 46, both shown in FIG. 3.

Needle element 20 is made of a super-elastic material with memory, such as nitinol. Needle element 20 has a predetermined rest configuration, usually straight. The application of an external force of a limited magnitude to the needle element 20 may deform it out of the rest configuration and into another configuration. Needle element 20 will however spring back to its straight or other predetermined configuration upon cessation or termination of the external force. This same operative functionality adheres to the device of the present invention, discussed below with reference to FIG. 16.

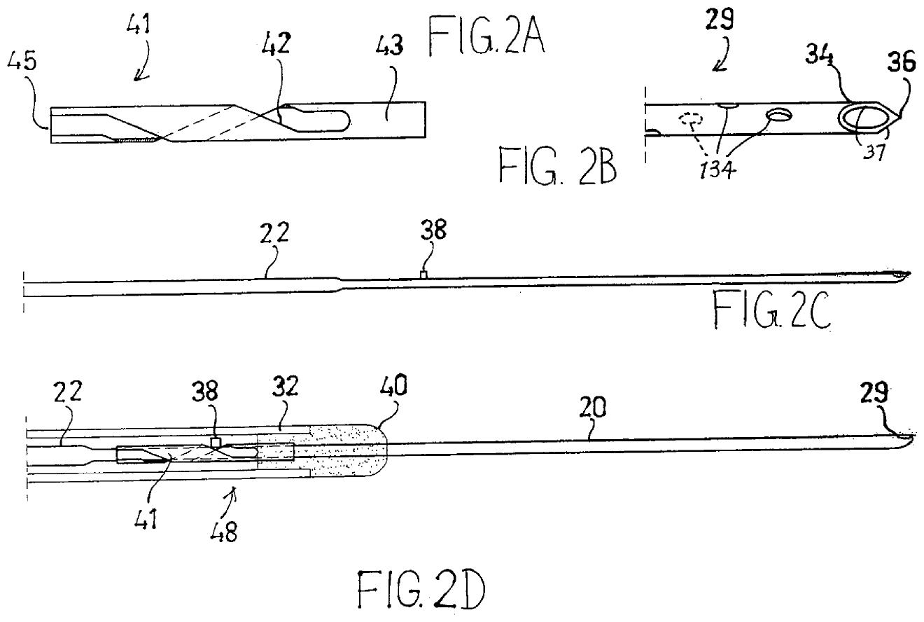

Sheath member 32 is made of a polymeric material such as PTFE (Teflon™), or PEEK. As shown in FIGS. 2A-2D, needle element 20 possesses a distal end configured in the shape of a spoon 34 with a sharp piercing tip or point 36. Spoon 34 may be provided with sharp curved lateral edges 37 for facilitating the cutting of internal body tissues during an endoscopic biopsy operation. Spoon 34 may be formed in part by beveling the end of the tubular shaft member. A camming mechanism may be provided at the distal end of sheath 32. The camming mechanism includes a key element or cam follower at the proximal end of needle element 20 in the form of a small laterally or transversely extending stub 38. Proximally to stub 38, needle element 20 widens into tubular shaft structure 22 also made of flexible material which may or may not be a similar super-elastic material such as that of needle element 20. Tubular structure 22 may be made of stainless steel or a shape memory material such as nitinol, same as needle element 20. Needle element 20 and tubular structure 22 are housed inside sheath 32. At the distal end of sheath 32 there is a metal collar 40 which is coupled with tubular member 32.

As shown in FIGS. 2A and 2D, a distal subassembly 48 includes a metal tube 41 having a spiral cutout or slot 42. Spiral cutout 42 terminates at the proximal end 45 of metal tube 41 and is spaced from the distal end 43 of metal tube 41.

Needle element 20 has a distal end 29 in the form of spoon 34 with the spoon's distal end tapering to a sharp tip or point 36.

FIG. 2D shows subassembly 48 assembled in its functioning configuration. As shown, stub 38 is fitted within spiral cutout 42 of metal tube 41. Metal tube 41 is coupled with and partially contained inside metal collar 40 and extends in a proximal direction into sheath 32.

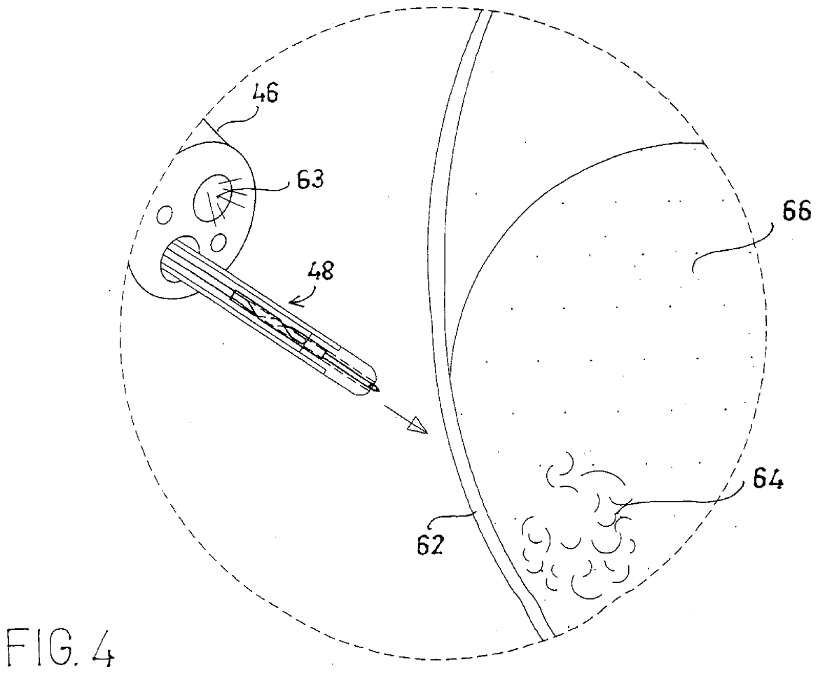

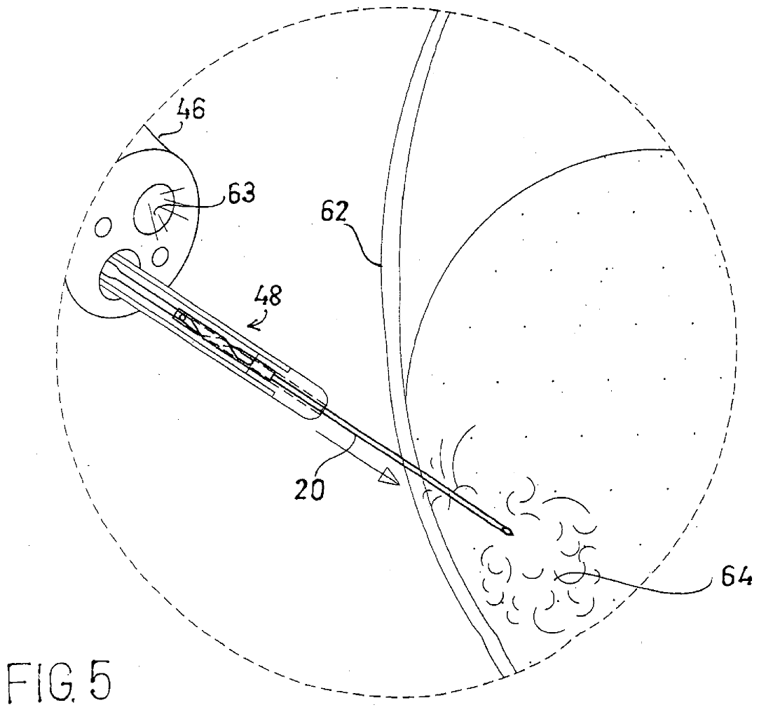

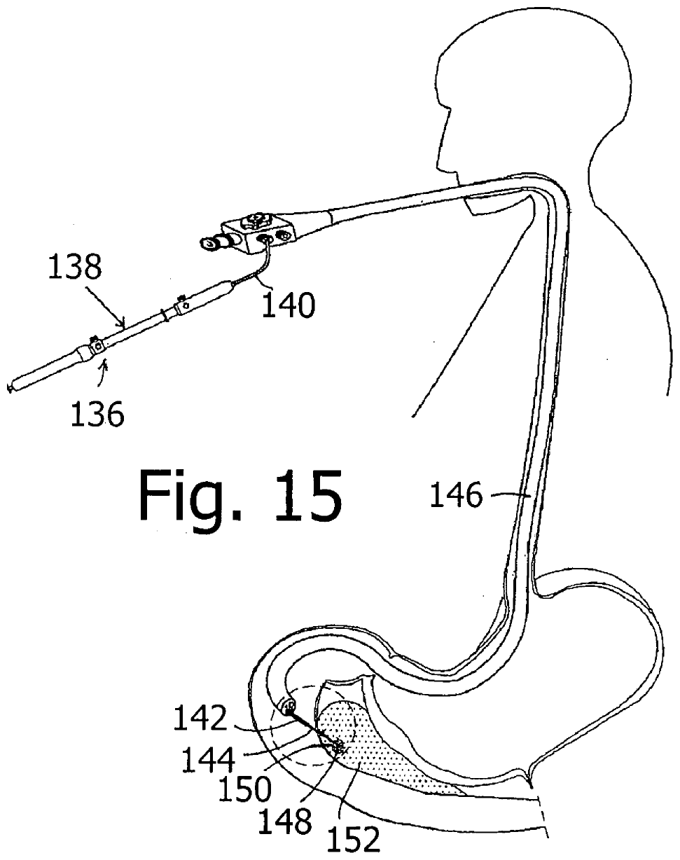

The configuration of distal subassembly 48 is designed to optimize the utilization of the instrument assembly of FIG. 1 in obtaining a core biopsy possibly indicative of a tumor growth from an internal body organ or tissue. As shown in FIG. 15, endoscope insertion member 46 is inserted through a patient's mouth, through the esophagus and stomach into the duodenum. As shown in FIG. 3, insertion member 46 is provided with optical elements such as a lens 60 to enable visual inspection of an inner wall 62 of the duodenum and a light source 63 to illuminate these internal body parts. As shown in FIG. 4, when a mass 64 is detected in the pancreas 66 with an ultrasound device (not shown) attached to endoscope 46, needle element 20 is ejected from subassembly 48 by manipulating actuator subassembly 26 (FIG. 1) in the distal direction. As shown in FIG. 5, needle element 20 is introduced through duodenal wall 62 in the direction of mass 64.

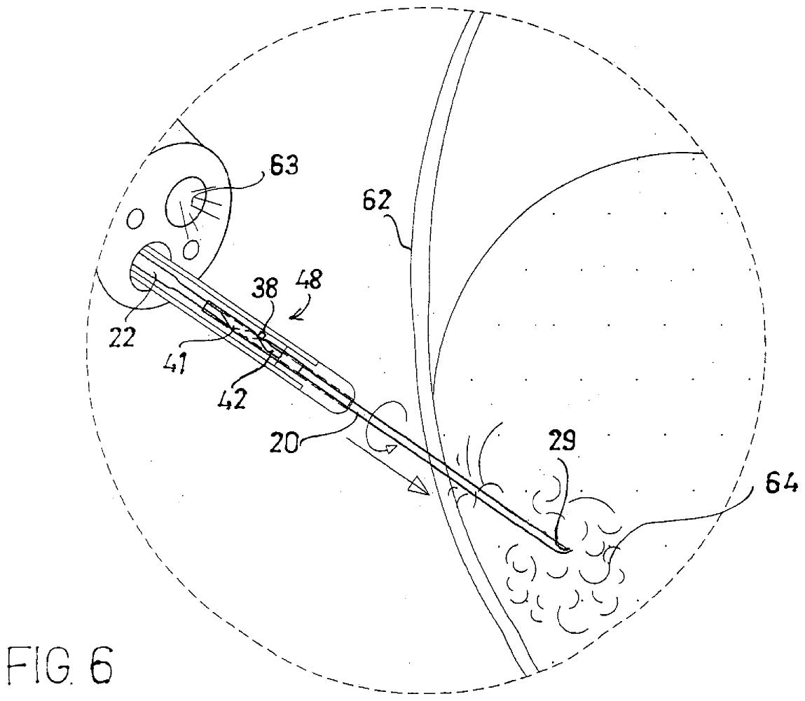

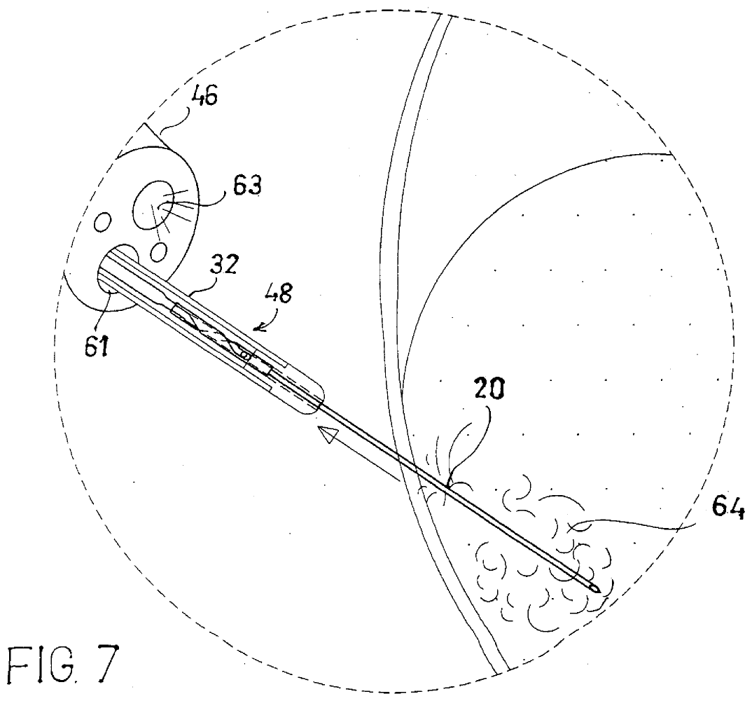

During an initial distal movement of needle element 20, stub 38 is spaced from slotted metal tube 41. As shown in FIG. 6, when needle element 20 reaches the interior of mass 64, illustrated in an ultrasound picture (not shown), continued pushing of needle element 20 engages stub 38 into spiral cutout 42 of metal tube 41. Further distal pushing of needle element 20 now induces needle element 20 to rotate inside tumor 64 due to a camming action of stub 38 against edges or surfaces of spiral cutout 42 in metal tube 41. This camming action causes the sharp spoon shaped distal tip or point 29 of needle element 20 and edges 37 to scoop out a core of tissue from tumor 64. Upon obtaining a core biopsy, a syringe (not shown) is attached to port 24 and operated to aspirate the tissue sample into the lumen of needle element 20. Once the aspiration procedure is completed, needle element 20 is retracted into sheath 32 (FIG. 7), and the entire assembly is withdrawn from biopsy channel 61 of endoscope 46. The specimen is then ejected into preservative by injecting fluid through port 24 into the lumen of tubular shaft member 22 and needle element 20.

The camming subassembly (tube 14, spiral cutout 42, stub or follower 38) is a structural arrangement for converting a linear motion partially into a rotary motion and may be located at any position along tubular shaft member 22, particularly inside actuator subassembly 26. Such an alternative design is depicted in FIGS. 8-11.

FIGS. 8-10 depict a handle or actuator assembly 70 of a core tissue sampling instrument that may be used in an endoscopic procedure utilizing optics alone or optics in conjunction with endoscopic ultrasonography. Such an instrument includes a flexible sheath member and a flexible shaft member as discussed above. Alternatively, handle or actuator assembly 70 may be part of a breast biopsy instrument having a substantially rigid tubular shaft member, or needle. Preferably, this would be a stainless steel needle. Handle or actuator assembly 70 includes an outer plunger handle or shifter 72 that telescopingly receives, in a distal end, a tubular plunger 74. Plunger handle or housing 72 is provided at a proximal end with a nipple or port element 76 for receiving a nozzle part of a syringe (not shown), while plunger 74 is provided at a distal end with a connector 78 for coupling to a tubular sheath member (not shown).

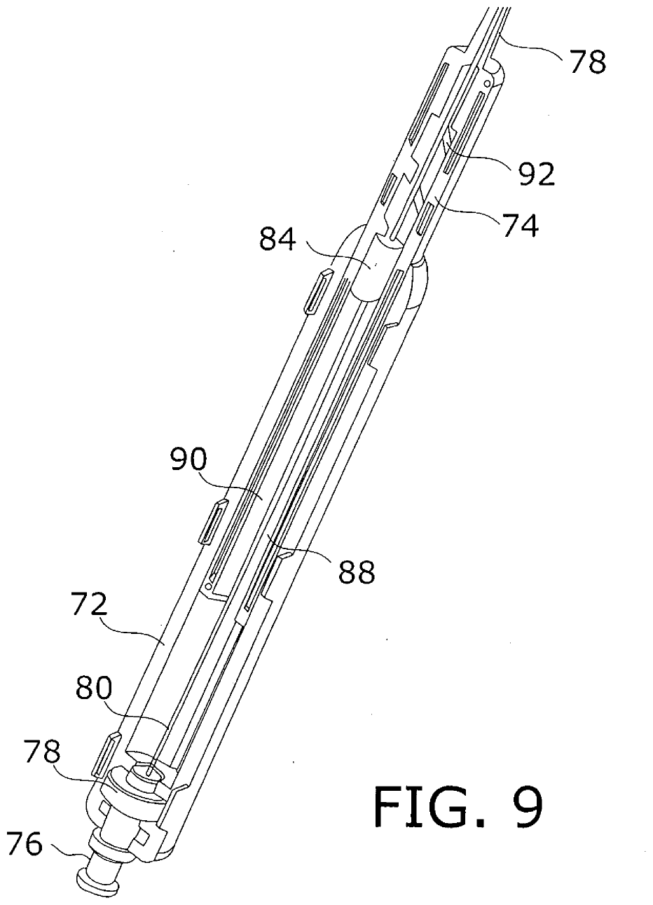

As shown particularly in FIG. 9, a tubular needle shaft 80 extends through connector 78, plunger 74, and handle or housing 72 and connects at a proximal end to a cap 82 rotatably seated in a cylindrical compartment 84 at the proximal end of plunger handle 72. Needle shaft 80 communicates with nipple or port element 76 via cap 82.

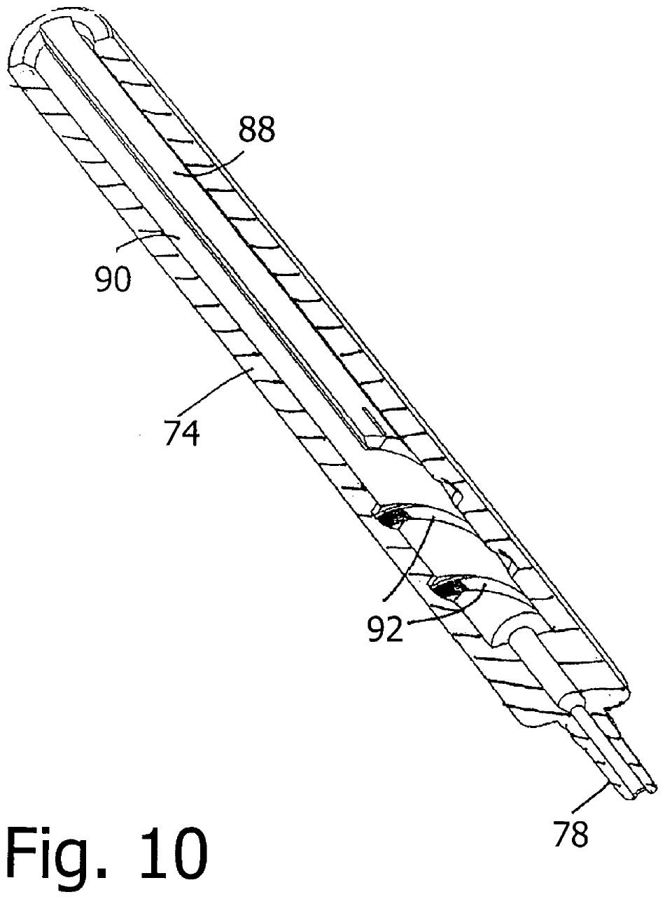

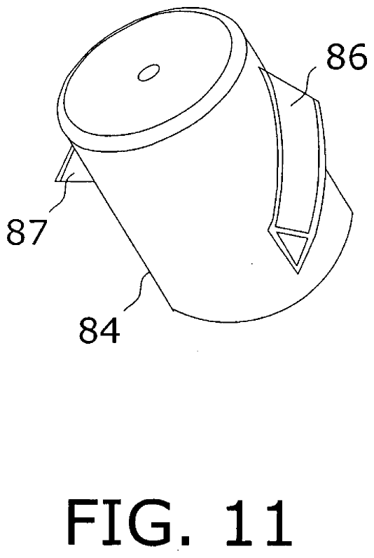

A cylindrical carrier or stylist 84 on needle shaft 80 exhibits a pair of outwardly projecting spiral rib or flange sections 86, 87 (FIG. 11) serving as keys or cam followers. Spiral rib or flange sections 86, 87 are substantially triangular in cross-section as shown in FIG. 11. A pair of linear grooves or keyways 88 (FIGS. 9 and 10) extend longitudinally along an inner surface 90 of plunger 74 and communicate or intersect at their distal ends with respective spiral cutouts or grooves 92 in inner surface 90 of plunger 74.

During a needle ejection procedure, an operator holds plunger 74 steady or fixed relative to a patient while moving handle or shifter 72 in a distal direction over the plunger. During an initial portion of this distally directed stroke of handle 72, rib or flange sections 86, 87 are each located in and guided by a respective groove or keyway 88 while needle shaft 80 translates distally. As this distal motion of handle 72 continues, rib or flange sections 86, 87 enter respective spiral cutouts or grooves 92 and are constrained to follow those cutouts or grooves, thereby imparting a rotational motion to carrier or stylist 84 and needle shaft 80. Cutouts or grooves 92 thus define camming surfaces, tracks or keyways that convert linear motion of needle shaft 80 at least partially into a rotational motion. At their proximal ends, spiral cutouts or grooves 92 communicate or intersect with linear grooves or keyways 88.

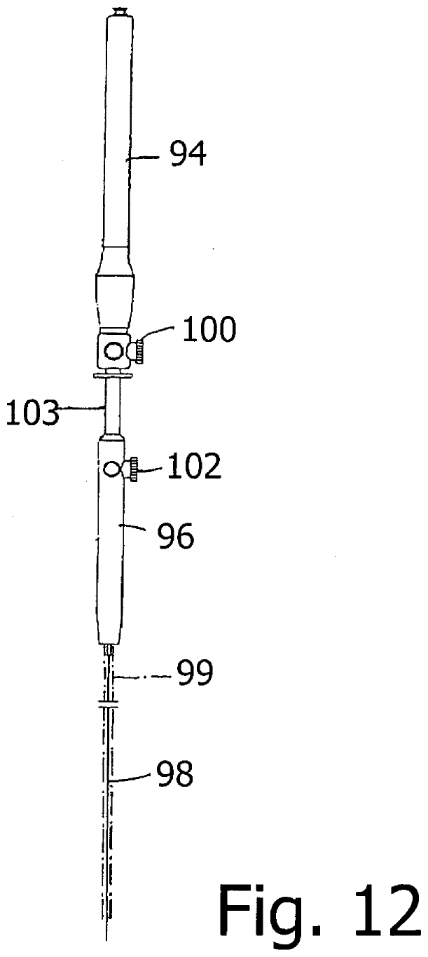

FIG. 12 depicts a modified core tissue sampling instrument that incorporates the internal functional elements of the embodiment of FIGS. 8-11. The instrument of FIG. 12 includes a handle or shifter member 94, a plunger member 96 and a tubular needle shaft 98 extending through a tubular sheath member 99. One or more set screws 100, 102 may be provided for fixing plunger 96 relative to handle 94. Plunger member 96 includes a tube 103 that is inserted into handle member 94. (As discussed hereinafter, tubular needle shaft 98 in the instrument assembly of FIG. 12 may be replaced by elongate shaft member 202 of FIG. 16.)

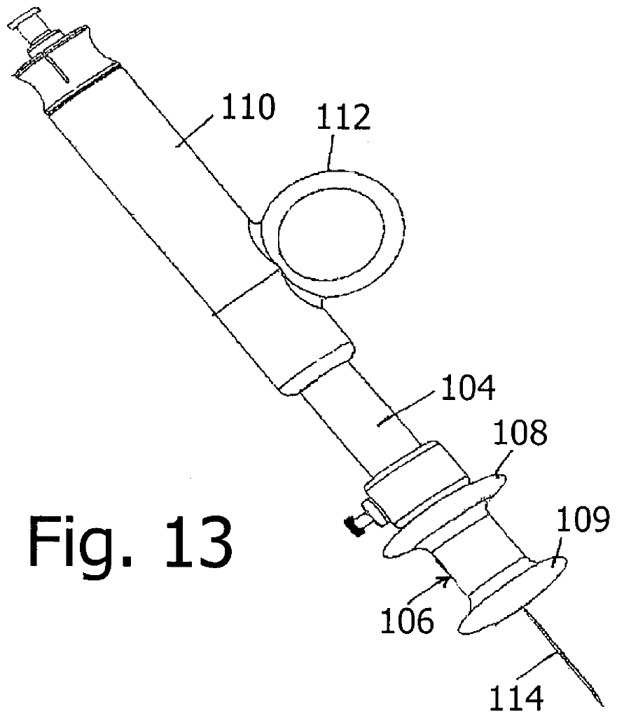

FIG. 13 depicts another variation of the core tissue sampling instrument of FIGS. 8-11 particularly useful for breast biopsies and core tissue sampling of other superficial organs. Here a plunger part 104 is provided with a spool 106 having a pair of opposed flanges 108 and 109 for receiving a forefinger and a middle finger of a user. An outer tubular handle or shifter member 110 is provided along a lateral surface (not separately designated) with a thumb ring 112. Finger spool 106 and thumb ring 112 enable a user to hold the device and control the movement of a hollow needle shaft 114 with one hand.

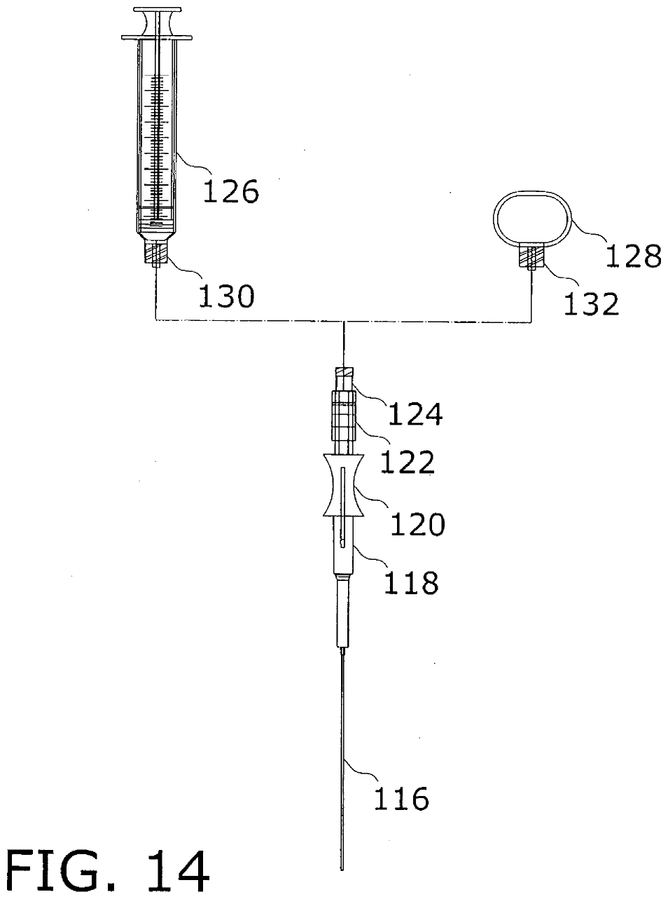

FIG. 14 shows yet another variation of the core tissue sampling instrument of FIGS. 8-11. A hollow needle shaft 116 extending through a tubular sheath member 117 is connected to a proximal end of a plunger part 118 that is provided with a finger spool 120. Plunger part 118 is slidably connected to a handle or shifter part 122 provided at a proximal end with an internally threaded screw connector 124 for removably coupling to a syringe 126 and alternatively to a thumb ring 128. Syringe 126 and thumb ring 128 are formed at their distal ends with externally threaded screw connectors 130 and 132 that mate with screw connector 124.

It is to be noted that any of the embodiments of a core tissue sampling instrument described and illustrated herein, particularly including the embodiments of 12-14, may be used with rigid needle shafts, e.g., shafts 98, 114, 116, in a percutaneous tissue sampling procedure. Such a procedure is followed, for example, to extract breast biopsy specimens. The needle shafts 98, 114, 116, etc., all have operative tips in the form of sharp needle points as discussed hereinabove particularly with reference to FIG. 2B. The sharp needle tips are preferably integrally formed parts of the respective needle shafts. In such percutaneous procedures, it is not necessary for the sampling instrument to include a sheath. Thus, sheath members 99, 115, 117 are omitted.

The percutaneous core tissue sampling instrument exemplarily of FIGS. 12-14 may be used for prostrate, thyroid, parathyroid, and perhaps liver and kidney biopsy extraction, as well as in obtaining breast biopsies.

Accordingly, a method for obtaining a core biopsy specimen from a superficial organ uses an ultrasound probe to localize and delineate a suspected mass lesion in an internal organ of a patient. The method additionally comprises inserting the needle tip portion of needle shaft 98, 114, 116 through the patient's skin, pushing the needle towards and into the mass lesion under ultrasound guidance, shifting actuator handle or shifter member 94, 110, 122 in a distal direction, and during that shifting engaging the associated camming mechanism to cause a tip of the needle to rotate 360 degrees within the mass lesion, thereby severing a tissue sample from the mass lesion. Subsequently, the tissue sample is aspirated deeper into the needle (e.g., by operating a syringe), the needle is removed from the tissue, and a liquid is injected through the needle, causing the core biopsy to be ejected into a preservative solution.

It is to be further noted that any of the embodiments of a core tissue sampling instrument described and illustrated herein may be provided with an electrical connector operatively linked to the needle element for enabling the transmission of a cauterization current to a target tissue site internal to the patient. The use of cauterization is described hereinabove with reference to FIGS. 1-7.

As illustrated in FIG. 2B, any of the needle tips disclosed herein may be provided along at least a portion of its length with a plurality of longitudinally spaced apertures 134 for the dispensing of a liquid solution into internal tissues. Apertures 134 may be staggered circumferentially about the needle.

FIG. 15 is a schematic view of an upper portion of a patient's digestive tract, showing a step in an endoscopic procedure utilizing a core tissue sampling instrument 136 having an actuator subassembly 138 (as shown in FIG. 12), a flexible tubular sheath 140 with a distal end portion 142, and a flexible tubular needle shaft 144. Sheath 140 with needle shaft 144 therein is inserted through a biopsy channel (not shown) in a flexible endoscope insertion member 146. Upon locating of a target biopsy site ultra-sonographically, the operator advances sheath 140 and needle shaft 144 in a distal direction through the biopsy channel so that distal end portion 142 of sheath 140 and a needle tip 148 emerge from the distal end of the endoscope insertion member 146. Needle shaft 144 is further advanced so that needle tip 148 is inserted into a target tissue mass 150 inside the patient's liver 152. Further details of this procedure are described hereinabove with reference to FIGS. 3-7.

As depicted in FIG. 16, a medical instrument or part 200 comprises an elongate shaft member 202 including a first or proximal elongate segment 204 made of stainless steel, a second or distal elongate segment 206 made of nitinol, and a connector element 208 made of nickel. Connector element 208 is bonded on one side to a distal end 210 of proximal elongate segment 204 and on an opposite side to a proximal end 212 of distal elongate segment 206. Distal elongate segment 206 is formed at a free end, opposite connector element 208, with an operative element such as a needle tip 216. For use in endoscopic biopsy or tissue sampling procedures as described above, the entire shaft member 202 has a diameter sufficiently small for insertion into, and disposition, in a channel of a flexible endoscope, for instance, into biopsy channel 61 of endoscope insertion member 46 (or 146), shown in FIG. 3 (or FIG. 15).

Elongate shaft member 202 may replace needle member 98 in the instrument assembly depicted in FIG. 12. In that case, as discussed above, shaft member 202 is disposable within elongate tubular sheath member 99, and the elongate tubular sheath member has a diameter sufficiently small so that the elongate tubular sheath member is disposable in biopsy or working channel 61 of endoscope 46 (or 146). Tubular sheath member 99 and elongate shaft member 202 are connected or mounted at least indirectly to handle 94.

Where elongate shaft member 202 is included as a component of an endoscopic tissue-sampling assembly as described above with reference to FIGS. 8-12, shaft member 202 is preferably tubular throughout, so that proximal elongate segment 204, distal elongate segment 206 and connector 208 are all tubular elements. Also proximal segment 204 and distal elongate segment 206 are necessarily flexible elements where the shaft member 202 is used in a flexible endoscopic tissue-sampling procedure. Proximal elongate segment 204 is preferably a tubular braid of stainless steel threads or stainless steel filaments wound in a coil or spiral, whereas distal elongate segment 206 is a solid or unitary nitinol tube. The two elongate segments 204 and 206 and connector 208 together exhibit a smoothly continuous internal lumen of uniform diameter, so as to easily receive or accommodate a central wire or stylet (preferably of nitinol). Likewise, the outer diameters of proximal elongate segment 204, distal elongate segment 206 and connector 208 are preferably equal, for facilitating unimpeded sliding motion through a surrounding sheath (e.g., sheath 99, FIG. 12).

Distal end segment 206 may be provided with a different tip configuration, other than a sharp needle tip 216, such as a scoop as described above. Ancillary features may be provided such as stippling 214 on an outer surface of distal segment 206, for facilitating detection of the instrument's distal end via ultrasound.

Although the invention has been described in terms of particular embodiments and applications, one of ordinary skill in the art, in light of this teaching, can generate additional embodiments and modifications without departing from the spirit of or exceeding the scope of the claimed invention. For example, it is to be appreciated that the tripartite connectivity of parts 204, 206, and 208 may be applicable in other kinds of medical instruments, such as guide wires and catheters. Guide wires are typically solid, rather than tubular members. Both guide wires and catheters are usually flexible. However the invention can also find application in devices that are partially rigid. For instance, a device might include a proximal end portion in the form of a tubular stainless steel rod or tube and a distal end portion in the forin of a flexible shape-memory nitinol rod or tube. The nickel connector in these cases may be solid or tubular, depending on the application.

It is to be appreciated that the present invention may find application as well in connecting two instrument components one of stainless steel and the other of nitinol, where the one component is made of stainless steel and the other component of nitinol that simple welding of the one component directly to the other is impossible.

Accordingly, it is to be understood that the drawings and descriptions herein are proffered by way of example to facilitate comprehension of the invention and should not be construed to limit the scope thereof.

What is claimed is:

1. A medical instrument comprising:
an elongate tubular sheath member having a diameter sufficiently small so that said sheath member is disposable in a channel of a flexible endoscope, said sheath member having an opening at a distal tip, said opening extending in a plane at least partially transverse to a longitudinal axis of said sheath member;
an elongate shaft member longitudinally passing through at least a substantial portion of said elongate tubular sheath member, said elongate shaft member including (i) a first elongate flexible tubular segment made at least predominantly of stainless steel, (ii) a second elongate flexible tubular segment made of nitinol, and (iii) a connector element of nickel, said connector element being bonded on one side to an end of said first elongate flexible tubular segment and on an opposite side to an end of said second elongate flexible tubular segment, said second elongate flexible tubular segment being formed at a distal or free end, opposite said connector element, with an operative element;
an actuator handle subassembly with a manually actuated shifter member operatively connected to a proximal end of said elongate shaft member at least for longitudinally moving said shaft member;
a camming subassembly operatively connected to said shaft member and to said shifter member at least for causing said elongate shaft member to rotate during an axial motion of said shaft member, wherein said camming subassembly comprises at least one key element and at least one corresponding guide element; and
wherein an axial force applied to said shifter member shifting said shifter member in an axial direction causes said shaft member including said operative element to advance in an axial direction, while concurrently effecting a rotation of said operative element.

2. The medical instrument defined in claim 1 wherein said operative element is a hollow needle tip.

3. A medical instrument comprising:
an elongate flexible tubular shaft member having a diameter sufficiently small so that said shaft member is disposable in a channel of a flexible endoscope, said elongate flexible tubular shaft member including:
a first elongate flexible tubular segment made of stainless steel;
a second elongate flexible tubular segment made of nitinol; and
a connector element made of nickel, said connector element being bonded on one side to an end of said first elongate flexible tubular segment and on an opposite side to an end of said second elongate flexible tubular segment.

4. The medical instrument defined in claim 3, further comprising an elongate flexible tubular sheath member, said elongate flexible tubular shaft member being disposable within said elongate flexible tubular sheath member, said elongate flexible tubular sheath member having a diameter sufficiently small so that said elongate flexible tubular sheath member is disposable in a channel of a flexible endoscope.

5. The medical instrument defined in claim 3 wherein said first elongate flexible tubular segment is a proximal end portion of said elongate flexible tubular shaft member, said second elongate flexible tubular segment being a distal end portion of said elongate flexible tubular shaft member.

6. The medical instrument defined in claim 3 wherein said connector element is heat bonded or welded on said one side to said first elongate flexible tubular segment and on said opposite side to said second elongate flexible tubular segment.

7. The medical instrument defined in claim 3 wherein said second elongate flexible tubular segment is provided at a free end, opposite said connecting member, with a needle configuration.

8. The medical instrument defined in claim 3 wherein said second elongate flexible tubular segment is provided at a free end, opposite said connecting member, with a spoon-shaped configuration.

9. A medical instrument comprising:
a handle;
a tubular sheath member connected to said handle and having a diameter sufficiently small so that said tubular sheath member is insertable inside working channel of a flexible endoscope; and
an elongate shaft member having a major portion disposed within said tubular sheath member, said elongate shaft member including:
an elongate proximal segment made of stainless steel;
an elongate distal segment made of nitinol; and
a connector element made of nickel, said connector element being bonded on one side to said elongate proximal segment and on an opposite side to said elongate distal segment.

10. The medical instrument defined in claim 9 wherein said connector element is heat bonded or welded on said one side to said elongate proximal segment and on said opposite side to said elongate distal segment.
